(19) United States
(12) Patent Application Publication
(10) Pub. No.: US 2022/0117531 A1
(43) Pub. Date: Apr. 21, 2022

(54) LIQUID-VOLUME MEASURING DEVICE

(71) Applicant: Coloplast A/S, Humlebaek (DK)

(72) Inventors: Henrik Jensen, Copenhagen NV (DK); Kasper Bertelsen, Copenhagen S (DK)

(21) Appl. No.: 17/273,339

(22) PCT Filed: Sep. 6, 2019
(86) PCT No.: PCT/DK2019/050264

§ 371 Date: Mar. 4, 2021

(30) Foreign Application Priority Data
Sep. 7, 2018 (DK) PA 2018 70578

(51) Int. Cl. A61B 5/20; A61B 5/00; A61B 8/00; A61B 8/08; G01F 15/075; G01F 1/66

(52) U.S. Cl. CPC A61B 5/208; A61B 5/0002; A61B 5/202; A61B 5/6874; G01F 1/662; A61B 8/5207; G01F 15/0755; A61B 8/488; A61B 8/4227

(57) ABSTRACT

The present invention relates to a hand-held volume measuring device configured for being clamped onto part of a urinary catheter and for measuring the volume of urine miming through the urinary catheter over a period of time.

### FIELD OF THE INVENTION

The present invention relates to a liquid-volume measuring device configured for measuring the volume of a liquid running through a urinary catheter.

### BACKGROUND OF THE INVENTION

In some urinary conditions, patients are required to use urinary catheters to be able to empty their bladder and/or equivalent. Sometimes, catheter users need to gather information relating to their urinary ouput. Such information may include the volume and/or color of the urinary output, as well as the frequency in which they need to use the urinary catheter.

For example, in some urinary conditions, the patients may not be able to feel when their bladder is full and when they have to empty it. Thus, especially in the beginning of their course of disease, such catheter users need to learn how often they need to empty their bladder, depending on their liquid input, the size of their bladder, etc. However, such urine output can be troublesome to obtain for users. At present, urine measuring devices often include a measuring cup of some kind, into which the user needs to empty the content of the urinary catheter, take read offs, and write down the information. Having to handle both the urinary catheter, the measuring device, a pen and a block of paper, as well as carry these objects around is very unhandy for users. Furthermore, the measuring device will have to be thoroughly washed afterwards.

Hence, an improved measuring device would be advantageous, and in particular a more handy measuring device, allowing the user to easily obtain the urinary output without having to carry around to many objects.

Healthcare professionals, such as medical doctors, may use such information to provide users with the best possible treatment and guidance. However, the users' urinary output is often kept confidential. Therefore, medical companies and hospitals do not have much user data relating to urinary output for different user groups.

Further, it would be advantegous if such urinary output data could be collected from users, such that the users could obtain better guidance from doctors and such that medical companies could improve their products to different types of users with specific needs, if given access to urinary output data from the individual users or aggregated users using the invention.

### Object of the Invention

An object of the present invention is to provide an alternative to the prior art. In particular, it may be seen as a further object of the present invention to provide a urine measuring device that solves the above mentioned problems of the prior art.

### SUMMARY OF THE INVENTION

Thus, the above described object and several other objects are intended to be obtained in a first aspect of the invention by providing a hand-held liquid-volume measuring device configured for being clamped onto part of a urinary catheter and for measuring the volume of a liquid running through the urinary catheter, the hand-held volume measuring device having a housing comprising
a channel running through the housing, which is shaped and dimensioned to be fitted around part of an outer surface of a urinary catheter
liquid-volume measuring means configured for measuring the volume of liquid running through the urinary catheter over a period of time;
user interaction means for activating and/or deactivating the liquid measuring means;
a wireless transmission means, which allows data from the liquid-volume measuring means to be transmitted to a personal terminal, such as a computer, a tablet, or a smartphone; and
an energy source, such as a rechargeable battery.

In some embodiments, the liquid-volume measuring means comprises
an ultrasound emitting unit, configured for emitting at least one beam or pulse of ultrasound through part of a urinary catheter
an ultrasound receiving unit, configured for receiving the at least one beam or pulse of ultrasound when it has been emitted through the urinary catheter.

In some embodiments, the device further comprises a processing unit, configured for receiving data from the liquid-volume measuring means and processing the data to obtain processed data including the volume of liquid running through the urinary catheter over a period of time.

In some embodiments, the wireless transmission means allows data from the liquid-volume measuring means and/or the processed data to be transmitted to a terminal, such as a computer, tablet, or smartphone.

In some embodiments, the housing further comprises a display unit, configured to display processed data from the processing unit, including the volume of a liquid running through the urinary catheter over a period of time.

In some embodiments, the housing has a longitudinal shape and less than 15 cm in all dimensions, such as less than 10 cm in all dimensions.

In some embodiments, the housing comprises a first portion and a second portion being pivotably connected to each other and each comprising a contact surface having an indentation running though the portion, shaped and dimensioned for partly receiving a urinary catheter.

Usually, the size (diameter, circumference) of urinary catheters, in particular intermittent urinary catheters, are from size 8 FR to size 18 FR. FR (or French size or Charriere (Ch)) is a standard gauge for urinary catheters approximately corresponding to the outer circumference in mm. More accurately, the outer diameter of the urinary catheter in mm corresponds to FR divided by 3. Thus 8 FR corresponds to a urinary catheter with an outer diameter of 2.7 mm and 18 FR corresponds to a urinary catheter with an outer diameter of 6 mm.

Usually, the length of urinary catheters, in particular intermittent urinary catheters, are adapted to the intended gender, such that male urinary catheters are typically at least 35 cm, such as between 35 cm and 50 cm, whereas female urinary catheters are typically between 70 mm and 150 mm.

In some embodiments, the device has an open configuration and a closed configuration, wherein
in the open configuration, the first portion and the second portion are only in contact with each other through a pivotable section,
in the closed configuration, the contact surface of the first portion and the second portion are in contact with each other such that the indentations running through the first and the second portion together enclose the channel which is adapted to be fitted around part of an outer surface of a urinary catheter.

In some embodiments, the device further comprises a locking mechanism, configured such that in the closed configuration, the first portion and the second portion are interlocked by means of the locking mechanism.

In a second aspect, the present invention relates to a system comprising
the handheld volume measuring device
a smartphone, tablet, or computer having an application software installed, programmed to receive and process data from the handheld volume measuring device,

wherein the application software is configured, based on input information and/or machine learning, to inform a user of the system of several statistic data, such as through a graphical user interface of the smartphone, tablet, or computer having the application software installed.

The statistic data may include how many times a day the user urinates, the average volume of urine each time they urinate, and the largest volume urinated so far, etc.

Preferably, a user can interact with the application software through a smartphone, tablet, or computer, and submit further data in the application software.

The application software may be programmed such that each user has a profile where they have to input basic information before they can use the application, such as name, country, gender, etc., as well as store data on body type+urinary catheter user+which brand+illness/type of condition.

The application may further be programmed such that data on intake (fluids+solids+medicine) can be input by the user. Such information could also be received from integrations with third party apps, e.g. fitness trackers (work-outs, sweat, etc).

Data received from the liquid-volume measuring means, as well as manually inputs by the user can be stored on the device (urine, condition of urine, leakage, etc.). As the volume and condition of the urine is a function of intake (solids, liquids, medicine) as well as physical exercise, which in turn means that if the user inputs that data in combination with the output, the application software will be able to process all data on one user and provide detailed overview on the individual user as well as aggregated data across all users using the invention.

The application software may be able to process data from the liquid-volume measuring device of the present invention, calculate urine output and store it. The application software may further be programmed to provide statistics, such as tables, graphs, and other forms of reporting on when, where, how much input/output the user has taken in/generated.

The application software may be programmed to learn cause/effect and help the user maintain an overview of timings of urinations, and predict best urination times based on intake, when and where, through machine learning.

The software application may be such that the user can extract data and customize reports which can be shared with doctors and other third parties. Furthermore, the application may be able to store aggregated data across all users and may be able to generate and share customized reports for medical research purposes.

The application software may further be programmed to send information from all users to a server or a cloud, such that all information is collected and can be used for further statistics. Such information could be used by hospitals or medical companies, e.g. to improve guidance from doctors and improve products for different types of users with specific needs.

The first and second aspect of the present invention may each be combined with any of the other aspects. These and other aspects of the invention will be apparent from and elucidated with reference to the embodiments described hereinafter.

### BRIEF DESCRIPTION OF THE FIGURES

The hand-held measuring device according to the present invention will now be described in more detail with regard to the accompanying figures. The FIGS. show one way of implementing the present invention and is not to be construed as being limiting to other possible embodiments falling within the scope of the attached claim set.

FIGS. 1A-1D illustrate an embodiment of a hand-held volume measuring device according to an embodiment of the present invention.

### DETAILED DESCRIPTION OF AN EMBODIMENT

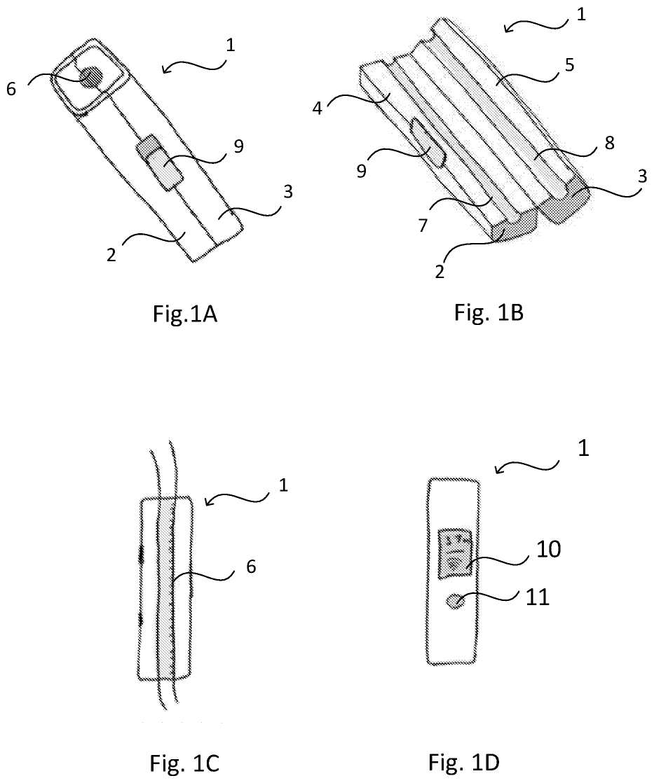

Reference is made to FIG. 1A illustrating an embodiment of a hand-held liquid-volume measuring device according to the present invention. The hand-held liquid-volume measuring device (also referred to as “the device”) of the present invention is configured for being clamped onto part of a urinary catheter and for measuring the volume of a liquid running through the urinary catheter over a period of time. The hand-held volume measuring device has a housing 1 comprising a channel 6 running through the housing 1. The channel 6 is shaped and dimensioned to be fitted around part of an outer surface of a urinary catheter.

The size of the channel 6 may vary, depending on the urinary catheter used by the specific user.

As illustrated in FIG. 1A, the housing 1 may have a longitudinal shape. Preferably, the device is less than 15 cm in all dimensions, such as less than 10 cm in all dimensions. In that way, the device is small enough to be easily transportated in a bag or a pocket, and small enough to be handled with one hand by the user. However, the device cannot be too small, as it has to contain different elements such as an energy source sizable enough to provide long term use. Preferably, the device is made from a material such as plastic, which allows the device to be dropped without breaking. Furthermore, the device has to be water resistant.

As illustrated in FIG. 1B, the housing 1 may comprise a first portion 2 and a second portion 3 being pivotably connected to each other, such as by a hinge of some kind. Both the first portion 2 and the second portion 3 comprise a contact surface 4, 5 having an indentation 7, 8 running through the portion 2, 3, shaped and dimensioned for partly receiving a urinary catheter. Thus, the indentation can, in a closed configuration of the device, have an inner diameter between 2.7 and 6 mm, reflecting common sizes of (intermittent) urinary catheters.

The device has an open configuration and a closed configuration. In the open configuration, which is illustrated in FIG. 1B, the first portion 2 and the second portion 3 are only in contact with each other through a pivotable section, such as a hinge. In the closed configuration, as illustrated in FIGS. 1A, 1C, and 1D, the contact surface of the first portion 4 and the contact surface of the second portion 5 are in contact with each other such that the indentations 7, 8 running through the first and the second portion together enclose the channel 6 which is adapted to be fitted around part of an outer surface of a urinary catheter. In that way, when the device is in the open configuration, the user can arrange a urinary catheter within an indentation of either the first portion or the second portion and close the device, by pivoting the contact surface of the first portion and the second portion towards each other. FIG. 1C illustrates a see-through view of a device according to an embodiment of the present invention, where part of a urinary catheter is arranged within the channel 6. Preferably, the device is configured such that it allows the user to secure the device in the closed configuration with one hand and to open the device with one hand. Thereby, the user would be able to measure the volume of urine running through a urinary catheter by clamping the device onto the urinary catheter before urinating and thereafter use the normal procedure to empty the bladder, using only one hand for holding the urinary catheter comprising the device. Furthermore, the device will not be in direct contact with urine, as it is clamped upon the outer surface of the urinary catheter. Thus, the user would in many instances not need to wash the device after use.

As illustrated in FIGS. 1A and 1B, the device may further comprise a locking mechanism 9, configured such that in the closed configuration, the first portion 2 and the second portion 3 are interlocked by means of the locking mechanism 9. Preferably, the locking mechanism 9 is a simple locking mechanism allowing the user to secure the device in the closed configuration with one hand and to open the device with one hand.

Furthermore, the hand-held liquid-volume measuring device comprises a liquid-volume measuring means configured for measuring the volume of liquid running through the urinary catheter over a period of time (not illustrated). Preferably, the liquid-volume measuring means uses ultrasound to measure the volume of urine running through the urinary catheter over a period of time and based on a series of measurements throughout the urinating period to measure time of flight and thus the flow of urine. Thus, the liquid-volume measuring means may comprise an ultrasound emitting unit, configured for emitting at least one beam or pulse of ultrasound through part of a urinary catheter and an ultrasound receiving unit, configured for receiving the at least one beam or pulse of ultrasound when it has been emitted through the urinary catheter. The volume of liquid running through the urinary catheter can then be calculated based on data from the liquid-volume measuring means, as well as the diameter of the urinary catheter and the density of the urine running through the urinary catheter. In some embodiments, the liquid measuring device is a ultrasonic flow meter.

Preferably, the device comprises wireless transmission means (not illustrated) allowing data from the liquid-volume measuring means to be transmitted to a terminal, such as a computer, tablet, or smartphone, which provides the processing power needed to calculate the volume of liquid running through the urinary catheter. The wireless transmission means may for example be wifi, Bluetooth, or infared connection means.

In some embodiments, the device itself comprises a processing unit (not illustrated), configured for receiving data from the liquid-volume measuring means and processing the data to obtain processed data including the volume of liquid running through the urinary catheter over a period of time.

If the device comprises a processing unit, the processed data may also be transmitted to a personal terminal, such as a computer, a tablet, or a smartphone via the wireless transmission means. The data from the liquid-volume measuring means and/or the processed data may also be transmitted or uploaded to a server or a cloud.

As illustrated in FIG. 1D, the device may further comprise an user interaction means 11 and a display unit 10.

The display unit 10 may be configured to display processed data, including the volume of a liquid running through the urinary catheter over a period of time.

The user interaction means 11 may be configured for activating and/or deactivating the liquid measuring means. The interaction means may for example be a button which activates the device, including the liquid measuring means when pressed and deactivates the liquid measuring means when released.

The device further comprises an energy source (not illustrated), such as a rechargeable battery, which provides power to the device, such as to the liquid-volume measuring means and the wireless transmitter.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### LIST OF REFERENCE SYMBOLS USED

1 housing

2 first portion of housing

3 second portion of housing

4 contact surface of first portion

5 contact surface of second portion

6 channel

7 indentation in first portion

8 indentation in second portion

9 locking mechanism

10 display unit

11 user interacting means

1. A hand-held liquid-volume measuring device configured for being clamped onto part of an intermittent urinary catheter and for measuring the volume of a liquid running through the urinary catheter, the hand-held volume measuring device having a housing (1) comprising a first portion (2) and a second portion (3) being pivotably connected to each other, the first portion (2) and the second portion (3) each comprising a contact surface (4, 5) having an indentation (7, 8) running through the portion (4, 5) being shaped and dimensioned for partly receiving an intermittent urinary catheter, the indentation, in a closed configuration of the device, having an inner diameter between 2.7 and 6 mm, the housing (1) further comprising
liquid-volume measuring means configured for measuring the volume of liquid running through the urinary catheter over a period of time;
user interaction means (11) for activating and/or deactivating the liquid measuring means;
a wireless transmission means, which allows data from the liquid-volume measuring means to be transmitted to a personal terminal, such as a computer, a tablet, or a smartphone; and
an energy source, such as a rechargeable battery.

2. A hand-held volume measuring device according to claim 1, wherein the channel has a diameter between 2.7 mm and 6 mm.

3. A hand-held volume measuring device according to any of claims 1-2, wherein the liquid-volume measuring means comprises
an ultrasound emitting unit, configured for emitting at least one beam or pulse of ultrasound through part of a urinary catheter
an ultrasound receiving unit, configured for receiving the at least one beam or pulse of ultrasound when it has been emitted through the urinary catheter.

4. A hand-held volume measuring device according to any of claims 1-3, wherein the device further comprises a processing unit, configured for receiving data from the liquid-volume measuring means and processing the data to obtain processed data inlcluding the volume of liquid running through the urinary catheter over a period of time.

5. A hand-held volume measuring device according to any of claims 1-4, wherein the wireless transmission means allows data from the liquid-volume measuring means and/or the processed data to be transmitted to a terminal, such as a computer, tablet, or smartphone.

6. A hand-held volume measuring device according to any of claims 1-5, wherein the housing further comprises a display unit (10), configured to display processed data from the processing unit, including the volume of a liquid running through the urinary catheter over a period of time.

7. A hand-held volume measuring device according to any of claims 1-6, wherein the housing (1) has a longitudinal shape and less than 15 cm in all dimensions, such as less than 10 cm in all dimensions.

8. A hand-held volume measuring device according to any of claims 1-7, wherein the housing (1) comprises a first portion (2) and a second portion (3) being pivotably connected to each other and each comprising a contact surface (4, 5) having an indentation (7, 8) running through the portion, shaped and dimensioned for partly receiving a urinary catheter.

9. A hand-held volume measuring device according to any of claims 1-8, wherein the device has an open configuration and a closed configuration, wherein
in the open configuration, the first portion (2) and the second portion (3) are only in contact with each other through a pivotable section,
in the closed configuration, the contact surface of the first portion (4) and the second portion (5) are in contact with each other such that the indentations (7, 8) running through the first portion (2) and the second portion (3) together enclose the channel (6) which is adapted to be fitted around part of an outer surface of a urinary catheter.

10. A hand-held volume measuring device according to any of claims 1-9, wherein the device further comprises a locking mechanism (9), configured such that, in the closed configuration, the first portion (2) and the second portion (3) are interlocked by means of the locking mechanism (9).
